(19) United States
(12) Patent Application Publication
(10) Pub. No.: US 2020/0116701 A1
(43) Pub. Date: Apr. 16, 2020

(54) METHODS FOR IDENTIFYING AGENTS WHICH INDUCE (RE) DIFFERENTIATION IN UN- OR DEDIFFERENTIATED SOLID TUMOR CELLS

(71) Applicants: Knud ESSER, Cologne (DE); Andrea KULIK, Munich (DE)

(72) Inventors: Knud ESSER, Cologne (DE); Andrea KULIK, Munich (DE)

(21) Appl. No.: 16/626,896

(22) PCT Filed: Jun. 28, 2018
(86) PCT No.: PCT/EP2018/067397

§ 371 Date: Dec. 27, 2019

(30) Foreign Application Priority Data
Jun. 29, 2017 (EP) 17178817.7
May 2, 2018 (DE) 20-2018-002-198.9

(51) Int. Cl. G01N 33/50; C12N 5/09; G01N 33/92; G01N 21/80; C12M 1/04; C12M 1/00; C12M 1/34; C12M 1/32

(52) U.S. Cl. CPC G01N 33/5011; C12N 5/0693; G01N 33/92; G01N 21/80; C12M 23/24; C12N 2501/315; C12M 29/00; C12M 41/26; C12M 23/12; C12N 2501/33; C12M 23/38

(57) ABSTRACT

The present invention comprises a test for the identification of an agent, which induces (re-)differentiation in a tumour cell based on the novel combined application of the two markers lactate as the end-product of the katabolic anaerobic glycolysis and neutral lipids as the end-product of the anabolic neutral lipid synthesis. The cell-based system is further characterized by a novel liquid handling procedure, which allows valid high throughput screening even over a longer time period of at least seven days, and a new viability test.

The present invention comprises a test for the identification of an agent, which induces (re-)differentiation in a tumour cell based on the novel combined application of the two markers lactate as the end-product of the katabolic anaerobic glycolysis and neutral lipids as the end-product of the anabolic neutral lipid synthesis. The cell-based system is further characterized by a novel liquid handling procedure, which allows valid high throughput screening even over a longer time period of at least seven days, and a new viability test.

Medium addition into and removal out of microtiter plates, respectively, can be performed via centrifugation as well as a new a system based on newly developed vessels. Cell viability can inventively be determined by the criterion of cell adherence of vital adherent cells for the first time after centrifugation non-invasively, marker-free and cost neutral, physically via absorption. Since reduction of cell adherence is specific for programmed cell death, the inventively method for determination of cell viability can for the first time be specifically used for the detection of apoptosis vs. necrosis. The method is specifically appropriate for high throughput screening. De- and undifferentiated cells, respectively, represent an important pathophysiological characteristic for tumour diseases.

Malignant tumour diseases represent a major health problem worldwide and are consecutively to cardiovascular diseases a major health issue in the industrialised countries. Along to information of the “Deutsche Krebsgesellschaft”, solely within Germany about 500.000 people fall ill every year due to cancer. Since most cancer diseases occur at increasing age, a further increase of incidences is expected due to the demographic change. Experts estimate that cases of cancer will increase up to 30 percent until 2050. Despite intensive research efforts and new molecular biological findings, the prognosis of many malignant tumours has only barely improved. Due to the high medical need for successful treatment options of hardly treatable tumour entities, the need for new agents is very high. In most cases the actual guidelines suggest treatment by chemotherapy, which does not allow a tumour specific therapy and comes along with many adverse effects for the patient.

Modern anti-cancer drug development aims at drugs having a low toxicity accompanied with a high specificity and anti-tumour effect. One class of such anti-cancer drugs are differentiation inducing agents, which are successfully applied in treatment of distinct forms of acute leukaemia. These drugs can, among other effects, activate cellular tumour defence mechanisms, which also correlate with a high cellular differentiation status, such as programmed cell death via apoptosis or an efficient antigen presentation. These agents possess a high potential to improve current anti-tumour therapy. However, in solid tumours, the mechanisms leading to a block of (re-)differentiation are only barely understood to date. New molecular biological investigations suggest that due the epigenetic expression pattern these tumours can be divided into more and less differentiated tumours, respectively. Among these, more differentiated tumours possess a better prognosis. Since in opposite to genetic mutations epigenetic changes are reversible, it is likely that specific differentiation inducing agents will also improve therapy in solid tumours. In this context it is especially interesting that differentiation inducing agents can develop a tumour subpopulation of high tumorigenicity, so called tumour stem cells, into less aggressive more differentiated tumour cells.

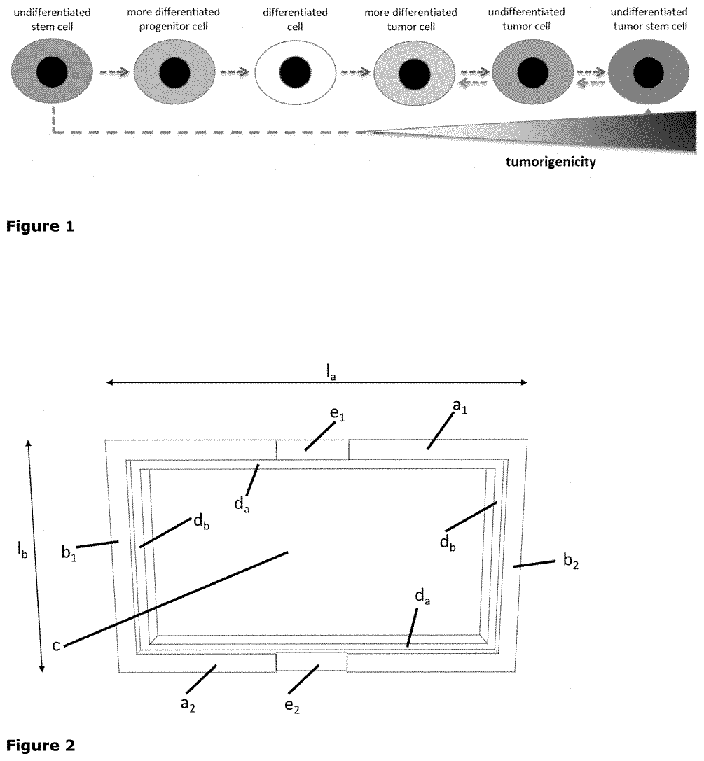

FIG. 1 shows a scheme for the development and tumorgenicity of tumour stem cells. These cells having a high stem cell character, probably define the malignancy of many tumours. Their development (arrows above) is not fully understood to date and probably very heterogenous. On the one hand in the context of tumour genesis they are discussed as the “Cell of origin”, that is supposed to be the cell which gives rise to the development of the tumour heterogenous cell forms. On the other hand, they are also discussed to develop from tumour cells with a low stemness, probably especially occurring under chemo therapy and determine the therapy success. It is known that differentiation inducing agents reduce the tumour stem cell stemness and develop it into a less malignant more differentiated tumour cell (arrows below). A lower stemness of tumour cells is associated with an increase in cellular tumour defence mechanisms (see above) as well as a reduced development of therapy resistance and a lower risk of relapse after therapy.

First studies with known agents, which have the potency to strongly alter the cellular gene expression pattern and to target distinct cellular differentiation blocks (for instance histone deacetylation inhibitors), suggest, that application of epigenetically acting agents has a high potency to also improve therapy in solid tumours like breast-, pancreas- and lung cancer. However, to date there are no anti-cancer drugs available on the pharmaceutical market which have specifically been developed for therapy of solid tumours by inducing differentiation which is likely due to the only rarely understood pathophysiology. There is a specifically lack of agents which specifically address pathophysiological mechanism which lead to a block of differentiation in solid tumours.

Current high-throughput screening systems which have been developed for the identification of (re-)differentiating agents in leukaemia or neuroblastoma address the expression profile of distinct genes, which have been described to be highly expressed in differentiated cells. However, due to the high heterogenicity of the gene expression profile in solid tumours this method is associated with a low specificity and high rate of false positive results. To reduce these characteristics, these systems require the parallel analysis of a higher amount of genes, which abolishes an efficient and broad application in high throughput screening. Alternatively, there are test systems available which analyse morphological changes and/or defined protein markers by high content imaging. These systems, however, are complex and cost intensive and require a very high logistical effort and high computer output for efficient analysis of the enormous data volumes. Therefore, these systems cannot be used for efficient and broad high throughput applications.

There is now a need for a method for the identification of agents inducing differentiation of a tumour cell and overcoming tumour specific differentiation blocks, respectively. This method should be characterized by a high specificity. Furthermore, the amount of false-negative results should be preferably low as well. Additionally, the system should be capable for high throughput testing which means a high amount of different substances can be investigated within a short time. In this context, also financial costs represent one relevant factor. The system should be economically priced. This includes a cell viability testing which is obligatory for high throughput screening of new compounds and is optimally implemented in the primary test, in a cost saving way. In case of the identification of agents which induce tumour, cell differentiation and overcome tumour specific differentiation blocks, respectively, it is of special interest to quantify cells, whose viability has been abolished due to apoptosis. The ability for programmed cell death via apoptosis is regarded as a feature of differentiated cells and can be induced via epigenetic (re-)programming in tumour cells (El-Metwally, T. H. & Pour, P. M. The retinoid induced pancreatic cancer redifferentiation-apoptosis sequence and the mitochondria: A suggested obligatory sequence of events. Journal of the Pancreas (2007)). The valid quantification of apoptotic cells in high throughput screening applications is extraordinary complex and connected to high financial costs (Hsu, K. W. et al. The application of non-invasive apoptosis detection sensor (NIADS) on histone deacetylation inhibitor (HDACI)-induced breast cancer cell death. Int. J. Mol. Sci. (2018). doi:10.3390/ijms19020452).

Surprisingly it could now be shown, that the valid identification of compounds inducing (re-)differentiation in a tumour cell can be made possible due to the novel combined quantification of lactate and neutral lipids. Depending on the model of tumorigenesis it is either referred to as redifferentiation (clonal evolution model) or differentiation (tumour stem cell model). The invented process includes, independently from the terminology mentioned above, such compounds which can induce the transformation of a tumour (stem) cell towards a less malignant (more differentiated) tumour cell or cell.

Furthermore, it has become surprisingly apparent that in the invented process, especially if performed at high throughput formats, the addition and the removal of various liquids (e.g. cell suspension, media, buffers, reagents, . . . ) from and to the wells of the used microplate (e.g. up to 1536-well format) can be performed via simple centrifugation in a (a) gentle, (b) simultaneous, (c) even, (d) bubble-free, (e) complete, (f) sterile, (g) efficient and (h) cost-neutral manner using the newly developed device consisting of partial devices (A) and (B). This device can be used with commercially available manual pipettes and does not necessarily require multichannel pipettes or complex robotic systems. All tasks, except for centrifugation, can be performed under sterile conditions using a commercially available cell culture bench.

Finally, a new method could be developed which physically and label-free measures the cell viability of adherently growing cells in microtiter plates via the criterion cell adherence after centrifugation by absorption measurement. The developed method can be used for the first time to determine the complete induction of apoptosis of the cells of interest in a cost-neutral, quick and valid manner and be applied in a high throughput system at the same time.

In a first embodiment the underlying object of the present invention is solved by a method for the identification of compounds inducing (re-)differentiation in un- or dedifferentiated cells, especially tumour cells, comprising:
a) provision of a cell culture sample consisting of de-/ or undifferentiated tumour cells,
b) bringing the compound of interest into contact with the cell culture sample
c) following the determination of the relative concentration of the first marker lactate in comparison to untreated cells, and
d) following the determination of the relative concentration of the second marker neutral lipids in comparison to untreated cells,
   wherein steps c) and d) may be performed in reverse order if necessary

Via the measurement of lactate and neutral lipids following the combined analysis of the results, the invented process enables for the first time the assessment whether a novel compound is capable of inducing (re-) differentiation in a cell and could therefore be used for the therapeutic treatment of tumour diseases.

For this invention tumour cells include so called tumour stem cells. Untreated cells are defined as cells that have not been treated with the compound of interest meaning they did not get physically in touch with the compound. For selected gene expression profiles, it must be considered that, due to the very high epigenetic heterogeneity of solid tumours, the examination of individually selected genes only insufficiently defines a (re-)differentiation in the tumour of interest. Metabolic processes which are known to occur in an intensified or reduced way in differentiated cells are less heterogenic altered in tumour cells and change towards a differentiated cell metabolic profile more conservedly upon tumour cell (re-)differentiation. Therefore, it could now have been shown, that when altering the tumour cell's metabolism in the direction to a differentiated cell's metabolism using a high throughput system for detection, a (re-)differentiation of degenerated tumour cells can be shown specifically and with less false positive results.

The present invention shows for the first time, that the combined quantification of the marker lactate and the marker neutral lipid, especially under anabolic and lipid reduced cell culture conditions, can be used to specifically identify (re-)differentiating compounds in solid tumours. Even if both markers have independently from each other been described as characteristic for cell differentiation, only the combined application enables a valid determination of (re-)differentiation particularly in tumour cells.

Recent investigations suggest that tumour cells can flexible cover their increased energy demand via beta oxidation and reduced anaerobic glycolysis without reducing malignancy (Porporato, P. E., Filigheddu, N., Pedro, J. M. B.-S., Kroemer, G. & Galluzzi, L. Mitochondrial metabolism and cancer. Cell Res. (2017). doi:10.1038/cr.2017.155), in contrast to the former assumption after which tumour cells conduct anaerobic glycolysis under aerobic conditions, a phenomenon known as the Warburg effect. In case of energy covering via beta oxidation, neutral lipids incorporated into lipid droplets serve as energy source for the tumour cell's increased energy demand (Cabodevilla, A. G. et al. Cell survival during complete nutrient deprivation depends on lipid droplet-fueled β-oxidation of fatty acids. J. Biol. Chem. (2013). doi:10.1074/jbc.M113.466656). Thus, the solitary determination of lactate is no adequate criteria for the determination of (re-)differentiation in tumour cells, but only when combined with an additionally increase of the cellular neutral lipid concentration towards untreated cells, can sufficiently be used to extrapolate to a (re-)differentiation.

Additionally, compounds for example solely inhibiting glycolysis can be excluded with the present invention.

Therefore, the novel combination of both markers is essential to the described test method.

It could be shown for the first time, that the addition of the strongest anabolic hormone insulin amplifies the changes of both markers in (re-)differentiated tumour cells essentially in the direction of an anabolic metabolism like described for insulin in non-tumour cells. Thus, the presence of insulin during the culturing of the cells as well as during step b) according to the invention is preferred.

In this context, the anabolic effect of an anabolic hormone can now be efficiently used for the first time to improve detection of (re-)differentiation via the both markers described. This is especially and inventively the case if the corresponding hormone receptor is only expressed on the (re-)differentiated cells of the (tumour-)cell of interest. This can know be utilised for the first time, e.g. with MDA-MB-231 cells which are negative for or poorly expressing prolactin receptor, in contrast to differentiated breast epithelial cells (Lopez-Ozuna, V. M., Hachim, I. Y., Hachim M. Y., Lebrun, J. J. & Ali, S. Prolactin pro-differentiation pathway in triple negative breast cancer: Impact on prognosis and potential therapy. Scientific reports (2016)). The addition of anabolic acting prolactin thereby only induces a change of the mentioned markers in the direction of an anabolic metabolism in (re-)differentiated cells. Via targeted incubation with/without hormone and in combination with quantification of the used markers lactate and neutral lipids it is due to the strong physiological specificity of the hormone-receptor binding for the first time possible to achieve a detection method for the analysis of prolactin receptor expression as a result of physiological receptor activation.

Furthermore, the analysis of the downstream signalling cascade following receptor binding can be covered. As a result, a conclusion on the expression of the physiologically functional receptor can be drawn. Comparably, functional expression of insulin receptor B and subtype A and B ratio, respectively, can be analysed by targeted incubation with/without insulin since in contrast to isoform A, which is predominantly expressed on non-differentiated cells, specific receptor activation only of isoform B, predominantly expressed on differentiated cells, induces a change of the mentioned markers in the direction of an anabolic metabolism in (re-)differentiated cells.

As especially low differentiated tumour cells, e.g. triple-negative breast cancer cells, mostly receive neutral lipids from extracellular sources (Antalis, C. J. et al. High ACAT1 expression in estrogen receptor negative basal-like breast cancer cells is associated with LDL-induced proliferation. Breast Cancer Res. Treat. (2010). doi:10.1007/s10549-009-0594-8) it could have been shown for the first time, that lipid-free or low in lipid cell culture conditions are of advantage to the increase of neutral lipids connected to (re-)differentiation in tumour cells. Since differentiated cells can switch their metabolic processes to anaerobic glycolysis under anaerobic conditions and in order to measure the increase in lactate production as a result of (re-) differentiation it has to be made sure that aerobic culture conditions are applied.

Both markers can directly or indirectly be quantified, e.g. via an enzyme activity correlating with metabolite concentration. The invented phenotypic method enables the implementation of so far unknown target structures, which represents an important technological advantage for the development of innovative drugs.

In accordance with the invented process, lactate and neutral lipids are used as markers for the identification of compounds inducing (re-)differentiation in a high throughput test system for the first time. In doing so, the test system can also be extended to other diseases than tumours, whose pathophysiology hints to cellular dedifferentiation or the blockage of differentiation playing a vital role. This could be the case for chronic inflammatory diseases like morbus chron, colitis ulcerosa or hepatitides including NASH (Non-alcoholic steatohepatitis), to name a few. Further examples would be degenerative diseases like arthrosis, Alzheimer's disease, amyotrophic lateral sclerosis (ALS) or various myopathies as well as various metabolic diseases like amyloidosis or lipidosis.

Besides the study of pathophysiological processes, the test system can be used to analyse physiological differentiation in healthy stem cells. In which case compounds specifically inducing physiological differentiation in healthy tissue could be identified, when combing the test system with healthy stem cells. This could play a role e.g. for the treatment of IRDS (infant respiratory distress syndrome).

Furthermore, the disease specific effect of a compound, meaning a specific targeting of and/or removal of a pathophysiological differentiation block, can be shown when using healthy stem cells. This way, it is possible to exclude an effect of the compound on healthy stem cells where no induction of differentiation is expected.

Accordingly, drug candidates inducing (re-)differentiation in solid tumours identified by the previously described test method could also be investigated for further therapeutic potential in the applications mentioned above.

During the physiological differentiation of stem cells, a decrease in anaerobic glycolysis and increase of oxidative phosphorylation for the use in energy generation takes place. This process represents an integral part of the cell's catabolism. Particularly under aerobic conditions, energy generation from oxidative phosphorylation prevails in differentiated cells. In contrast, an increase of anaerobic glycolysis under aerobic conditions is described within a vast number of tumour cells.

In the invented process, a reduction of anaerobic glycolysis under aerobic conditions is used as a metabolic indicator for identifying (re-)differentiation of solid tumour cells within a (high-throughput) screening system for the first time. Quantification of anaerobic glycolysis is made possible via measurement of lactate concentration. Generation of lactate can be determined by a change of pH value making an indirect concentration measurement via an indicator possible. Changes in pH can preferentially and innovatively be referred to anaerobic lactate production using bicarbonate buffered cell culture media: Due to the constant high partial CO₂ pressure of the CO₂-atmosphere provided by the incubator, which determines dissolved CO₂ concentration in the liquid, the comparably low amounts of CO₂ produced by aerobic degradation of energy stores is passed on to the equilibrium (CO₂ (liquid)<->CO₂ (gas)) leading to no pH changes in the medium. Thus, pH changes are negligibly influenced by cellular CO₂-production and inventively correlate much more precise with anaerobic lactate production cultured in bicarbonate buffered media (incubated in an appropriate CO₂-atmosphere, e.g. 5-10% CO₂ at 1.5-2.2% bicarbonate) compared to non-bicarbonate buffered media.

Alternatively, but preferred the amount of lactate produced by the cells can also be enzymatically measured by commercially available test systems. Alternatively, but preferred the activity of cellular lactate dehydrogenase (LDH) can be analysed in order to quantify the marker lactate. The activity of cellular LDH also correlates with anaerobic glycolysis and the formation of lactate.

Additionally and surprisingly, it has become apparent that especially under anabolic cell culture conditions (e.g. in the presence of insulin) the storage of neutral lipids in addition to lactate represents a suitable second marker for the verification of (re-)differentiation in solid tumours. This verification can be made using appropriate fluorescent ligands. For example, with the commercially available ligand BODIPY® 493/503 whose nonpolar structure specifically binds to neutral lipids. Particularly in lipid-free or low-in-lipid media the significance is especially visible so that the invented process preferably takes place in lipid-depleted or lipid reduced media.

Both, lipid-depleted and lipid reduced media are based on commercially available chemically defined basal media which is free of proteins, growth factors and lipids as provided by the manufacturer. Depending on the respective cell line either no lipid source is added (lipid-depleted media) or the basal media is supplemented with a lipid source of defined concentration (lipid-reduced media).

The invented process can be performed with only a few samples. However, the process can also be performed as a High-throughput screening (HTS)-system processing a variety of microtiter plates simultaneously. High-throughput screening is defined as previously described by Szymanski et al. (Adaption of High-Throughput Screening Drug Discovery—Toxicological Screening Test; Int. J. Mol. Sci 2012, 13, 427-452). The invented process is suitable for all screening modes as mentioned there in table 2 starting with Low-throughput screening up to Ultra-high throughput screening. However, the invented process provides for the first time a High-throughput screening or even Ultra-high throughput screening.

The conduction of the invented process takes place, particularly preferred, in microtiter plates. The usage of appropriate microtiter plates enables the implementation of the invited process as HTS-system. Thus, for example a 384-well microtiter plate can be used to analyse 384 samples at once within a short period of time.

As circumstances require, microtiter plates of other formats can naturally be used. The size of the microtiter plates is limited by the possibility of analysing the concentration of both markers. As far as the analysis takes place via optical spectroscopy, which can be conducted within a short period of time, High-throughput screening can be performed enabling a quicker identification of a compound. Equally, different cell culture samples can be investigated regarding the identification of a compound whereby the cell culture samples for example consist of different tumour cells. Accordingly, the invented process enables not only the identification of a compound but also the verification of compounds having a known effect in a known tumour for effects in other tumour entities.

Preferably, the microtiter plate is sealed with a gas permeable sealing foil during the invented process. This prevents liquid, solid or gaseous impurities from penetrating the individual samples and enables an even gas exchange via diffusion.

For the invented process a cell culture probe must be provided. This probe consists of the tumour cells which should react to the added compound. Subsequently the compound is added while cell culture persists. For this purpose, the addition of the commonly used media- or buffer components is required. With a particularly preferred embodiment the addition of and removal of media or buffer-components is made by an inventively partial device (A) and/or (B).

The preferred embodiment of the invented process includes the following steps:
a) provision of a cell culture sample consisting of de-/ or undifferentiated tumour cells,
b) bringing the compound of interest into contact with the cell culture sample
c) following the determination of the relative concentration of the first marker lactate in contrast to untreated cells, and
d) following the determination of the relative concentration of the second marker neutral lipids in contrast to untreated cells,
   wherein steps c) and d) may be performed in reverse order if necessary and wherein the addition of media and/or buffer components is made via partial device (B) and/or the removal of media and/or buffer components is made via partial device (A).

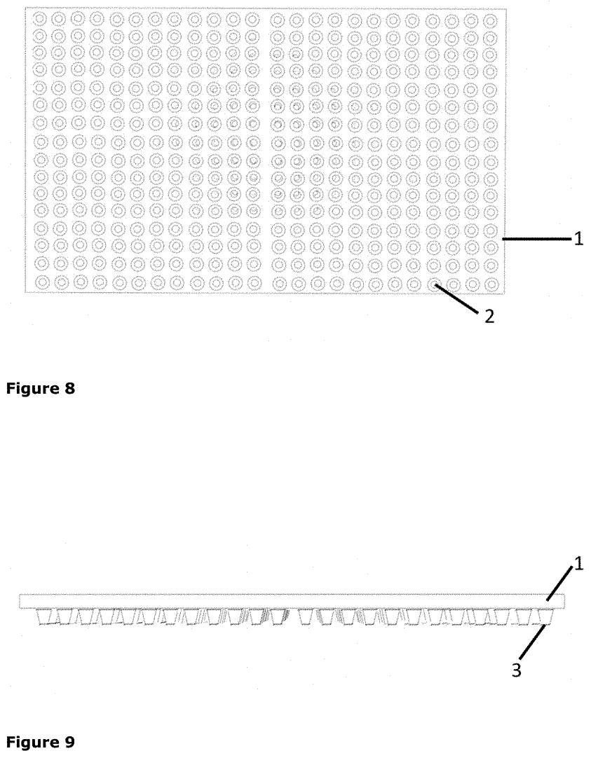

Partial device (A) and partial device (B) are shown schematically in the Figures. FIGS. 2 to 7 show partial device (A), whereas FIGS. 8 to 11 show partial device (B). The figures depict the different views, namely:

FIG. 2: Top view of partial device (A)

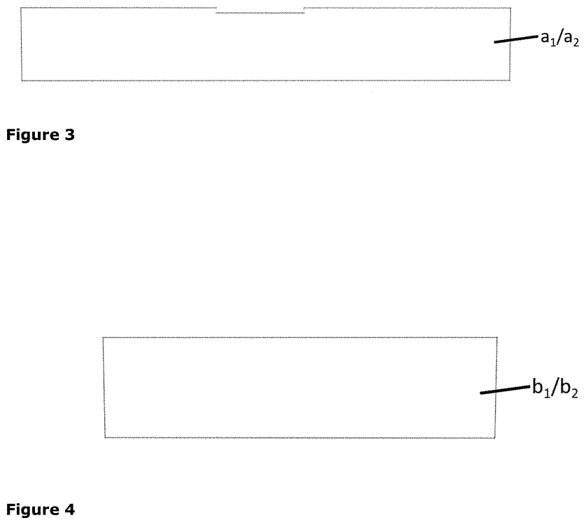

FIG. 3: Lateral view of partial device (A) (long side)

FIG. 4: Lateral view of partial device (A) (transverse side)

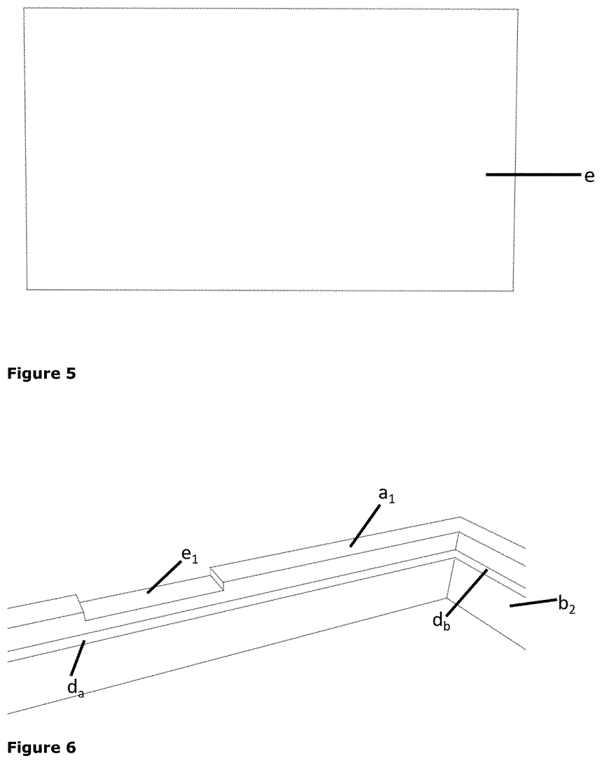

FIG. 5: Bottom view of partial device (A)

FIG. 6: Perspective view of partial device (A) illustrating the contact surface for the microtiter plate as well as the recesses for the simplified removal of the microtiter plate

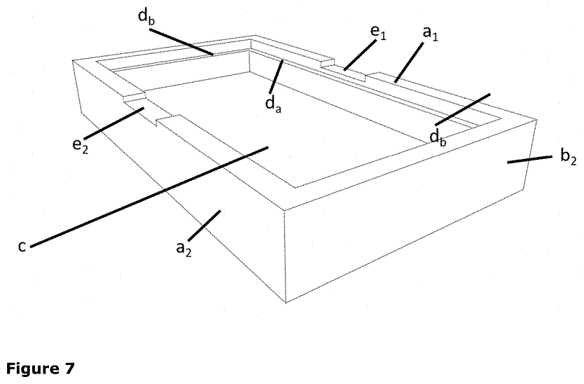

FIG. 7: Perspective view of partial device (A) as seen from the corner

FIG. 8: Top view of partial device (B)

FIG. 9: Lateral view of partial device (B) (long side)

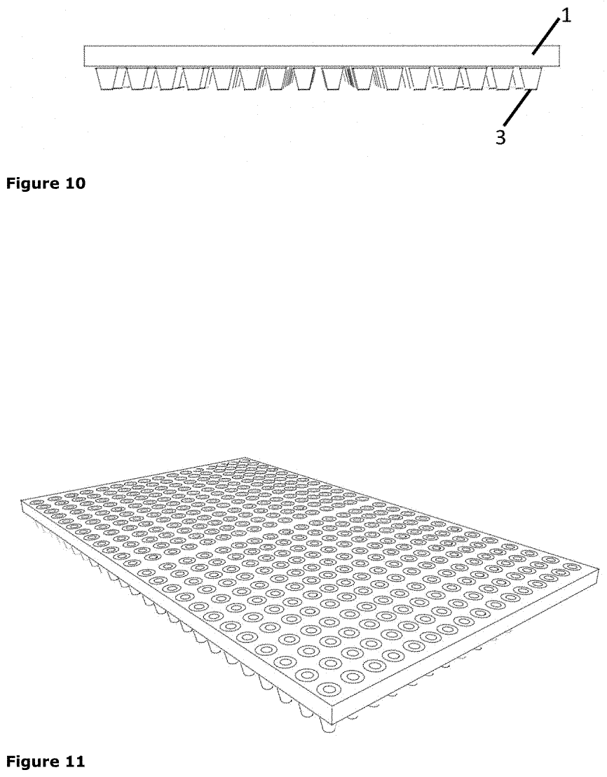

FIG. 10: Lateral view of partial device (B) (transverse side)

FIG. 11: Perspective view of partial device (B) as seen from the corner

The present invention thus refers in a further embodiment to a vessel for the removal of liquids from cells via centrifugation (partial device (A)) characterized by comprising:
4 side walls (a₁, a₂, b₁, b₂), where the two opposing side walls have the same length (lₐ, l_{b}), so that a rectangular shape is obtained,
a flat bottom (c) being connected in such a way with each of the side walls over all of the connecting area in a liquid proof way,
each of the side walls having a protrusion (dₐ, d_{b}) directed towards the inside of the vessel,
2 of the 4 side walls being opposite to each other having recesses (e₁, e₂) positioned in the middle of the lengths lₐ of the side wall on the upper surface of the respective side wall.

In a further embodiment, the present invention discloses a microtiter plate (partial device (B)) for culturing cells enabling addition of liquid via centrifugation, said plate comprising a surface (1) and wells (2), said wells being tapered towards the bottom (3) where an opening, especially a circular opening, is present. Said opening at the bottom (3) of the microtiter plate being adjusted in such way that the diameter of the opening is proportional to the surface tension of a liquid inside each of the wells and adjusted in such way that liquid can escape only under influence of forces stronger than the gravitational force.

Said partial devices (A) and (B) will be described in the following together with the inventive method.

Inventively, in order to determine markers mentioned in steps c) and d) the cell culture samples can be washed and/or one or more medium changes can be performed. For instance, this step can be performed using known pipetting methods. Inventively preferred, this step is made via centrifugation.

In contrast to common (multi-) pipetting systems aspirating the liquids, the invented process prefers to remove serum-containing media or low in-serum media or serum-free media from the single wells of a microtiter plate before the addition of compounds or fluorescent ligands via cost-effective, brief centrifugation.

Therefore, a vessel (partial device (A)) was developed for the use with commonly available centrifuge hangers. Microtiter plates are incorporated into the vessel with the opening of the wells pointing downwards into the vessel lumen whereby the microtiter plates are fixated by the container walls. As a result, the liquids from the single wells can be removed into the cavity provided by the container at the same time (FIG. 2-7; partial device (A)). This makes the efficient, gentle, sterile and complete removal of the liquids from the single wells possible in contrast to known pipetting methods using aspiration to remove liquids which often results in the loss of adherently growing cells and/or leads to liquid residues permanently remaining in the well. If needed partial device (A) can be installed together with various pipetting robots.

For assays using cell culture techniques based on adherently growing cells the media can be completely removed and collected after a few seconds of centrifugation (e.g. 100-300×g) whereby no liquid residues (residual volume) remain in the microplate wells. Thus, the efficient reduction and/or prevention of washing steps is made possible further resulting in shorter test durations and less amounts of washing solutions. A reduction in viability of the cells/cell lines in following cell culture trials could not be observed.

The efficient removal of media and the prevention of washing steps significantly increase the robustness of cell based assays. Furthermore, the efficient removal of supernatant via the partial device (A) and centrifugation, in contrast to using various aspiration attachments, also leads to a reduction/prevention of washing steps, enables efficient liquid removal without residues and prevents artefacts from occurring in non-cell based assay systems (e.g. ELISA-based assays).

Conventionally, the simultaneous addition of media or liquid to all wells of a microtiter plate can be realised via multichannel robotic heads (e.g. 96 to 1536 channels). Alternatively, it could be shown for the first time that this can also be done using commonly available multi-/single channel pipettes or single-/multichannel robotic heads (e.g. 1-16 channels) in combination with a specially designed microtiter plate (FIG. 8-11; partial device (B)).
In a first step the specially designed microtiter plate, consisting of wells slightly tapered towards the bottom where a (e.g. circular) opening has been built-in is used to add the liquids to be added. Every single well can be individually filled with liquids. Due to the surface tension the fluids will not flow through the e.g. circular opening at the bottom of the single wells slightly tapered towards the opening at the bottom. Here, the diameter of the drill hole must be adjusted to the surface tension of the liquid. Low surface tensions, e.g. due to the presence of detergents, require a smaller diameter. Liquids with higher surface tensions enable a bigger diameter. Alternatively, the filling of the microtiter plate consisting of wells slightly tapered towards the opening at the bottom can be done in one step via the full immersion of the microtiter plate into the liquid.
In a second step the microtiter plate, consisting of wells slightly tapered towards the (e.g. circular) opening at the bottom and filled with the liquid that should be transferred into a commercially available microtiter plate, is appropriately set onto the microtiter plate whereby the wells slightly tapered towards the opening at the bottom do not get into contact with the wells of or the liquids in the wells of the commercially available microtiter plate. To transfer the liquid under sterile conditions a fitting cover lid is added to partial device (B). Now, the transfer of the liquids from the microtiter plate, consisting of wells slightly tapered towards the opening at the bottom to the commercially available microtiter via centrifugation plate can take place. In doing so the liquids will automatically and independently from the applied rotational speed and at the lowest possible pressure be pressed from the wells of the microtiter plate which are slightly tapered towards the opening at the bottom to the wells of the commercially available microtiter plate. The reason this process is independent from the applied rotational speed of the centrifuge is, as at the moment when the radial force becomes stronger than the surface tension retaining the liquid in the wells slightly tapered towards the opening at the bottom, the liquids will be transferred into the wells of the commercially available microtiter plate. A further increase in rotational speed may lead to total transfer of liquid residues from the wells of the microtiter plate which are slightly tapered towards the opening at the bottom which have been left due to adhesion. This allows the perforated microtiter plate to be used for further applications without any cleaning steps needed in between.

In sum it has been shown that through the usage of partial device (B) together with a commercially available cell culture centrifuge and an applied rotational speed increasing from zero to up to e.g. 300×g single liquids can be transferred into the single wells of a microtiter plate in a targeted, gentle, simultaneously, even and complete as well as cost-efficient way.

Furthermore, it has been shown, that using centrifugation and the mentioned partial device (A) a new, cost-efficient viability test for adherently growing cells can be integrated in the field of cell culture using microtiter plates.

The preferred embodiment of the invented process further includes the following steps:
e) Determination of cell viability of adherently growing cells via quantification of absorbance after centrifugation, and
f) Determination of complete induction of apoptosis in the cells among a complete signal reduction to background level

In sum, the preferred embodiment of the invented process includes the following steps:
a) provision of a cell culture sample consisting of de-/ or undifferentiated tumour cells,
b) bringing the compound of interest into contact with the cell culture sample
c) following the determination of the relative concentration of the first marker lactate in contrast to untreated cells, and
d) following the determination of the relative concentration of the second marker neutral lipids in contrast to untreated cells,
   wherein steps c) and d) may be performed in reverse order if necessary
e) Determination of cell viability of adherently growing cells via quantification of absorbance after centrifugation, and
f) Determination of complete induction of apoptosis in the cells among a complete signal reduction to background level.

The particularly preferred process includes the following steps:
a) provision of a cell culture sample consisting of de-/ or undifferentiated tumour cells,
b) bringing the compound of interest into contact with the cell culture sample
c) following the determination of the relative concentration of the first marker lactate in contrast to untreated cells, and
d) following the determination of the relative concentration of the second marker neutral lipids in contrast to untreated cells,
   wherein steps c) and d) may be performed in reverse order if necessary
e) Determination of cell viability of adherently growing cells via quantification of absorbance after centrifugation, and
f) Determination of complete induction of apoptosis in the cells among a complete signal reduction to background level
   wherein
   the addition of media and/or buffer components is made via partial device (B) and/or
   the removal of media and/or buffer components is made via partial device (A).

Surprisingly, it has become apparent that using centrifugation and the mentioned partial device (A) a new, cost-efficient viability test for adherently growing cells can be integrated in the field of cell culture using microtiter plate.

Hereby, cell viability of adherently growing cells can be determined via the viability criteria cell adhesion measured by the non-invasive physical determination of absorption in commercially available microtiter plates (e.g. polystyrene-well bottom without any changes in the plastic surface). This allows a valid, rapid, non-invasive and label-free normalization of distinct measurements per microtiter plate well to the amount of viable cells.

The label-free quantification of vital cells can be performed due to the characteristics of cellular nucleotides and/or aromatic amino acids to physiologically absorb light at an absorption maximum from 260 nm to 280 nm (absorption maximum DNA/RNA: 260 nm; absorption maximum proteins: 280 nm). In case of measuring proteins this could be made possible for the first time, because the complete removal of media by centrifugation via partial device (A) leaves the wells of the microtiter plate free from media components such as proteins or buffer components which would otherwise affect the measurement. As an alternative to light absorption, light diffusion or cellular auto fluorescence can be quantified. Furthermore, a method using fluorophores or chromophores interacting with or incorporated by the cells prior to measurement can be used in which case the amount of remaining vital cells can be quantified in relation to background signal. As an alternative, the number of vital cells remaining can be determined via (high content) microscopy. The method can be performed as end-point measurement or can be implemented in defined kinetics by set medium changes and measurements prior addition/after removal of media. If cells are seeded at a density considerably lower than the density at which cell growth is reduced due to contact inhibition, kinetic trials for the determination of proliferation can also be performed.

As a lysis of cell is not implemented within this non-invasive, label-free method, viable cells are directly available for further studies. The described method can be used for toxicity studies as well as immunological toxicity assays. In the latter case, non-adherent immune cells are removed by centrifugation and therefore do not disturb the absorption measurement.

As, in contrast to necrotic cells, a loss of cell adherence is characteristic for apoptotic cells resulting in a reduced attachment to plastic surfaces (Kwon, H.-K., Lee, J.-H., Shin, H.-J., Kim, J.-H. & Choi, S. Structural and functional analysis of cell adhesion and nuclear envelope nano-topography in cell death. Sci. Rep. (2015). doi:10.1038/srep15623) a method was developed which correlates the reduction of optical absorption to background level after centrifugation with the complete induction of apoptosis in cells previously treated with the compound of interest. Thereby, non-/low-adherently cells are removed via the radial force generated by centrifugation which results in a lower optical absorption in the microplate wells to be examined. The complete detachment of originally adherent cells correlates with an induction of apoptosis in all cells at any time point measured due to the non-reversible detachment of the cells after the termination of cell adherence induced by apoptosis. The same applies to apoptotic vesicles, a later step in apoptosis occurring after cell detachment. This method provides a new, very efficient, non-invasive, label-free, valid and common apoptosis marker independent way to determine apoptosis which is, especially in high throughput applications, a due to complex kinetics difficult to measure variable.

Measuring absorbance values at background level after centrifugation indicates, independently form the time point of measurement, that absolute induction of apoptosis has occurred in the cells of interest in the respective microplate well(s). As the ability of cells to undergo apoptosis represents a distinctive feature of (re-)differentiation (El-Metwally, T. H. & Pour, P. M. The retinoid induced pancreatic cancer redifferentiation-apoptosis sequence and the mitochondria: A suggested obligatory sequence of events. Journal of the Pancreas (2007)), the quantification of apoptosis especially with kinetics and/or serial dilutions can be very well combined with the previously described markers lactate and/or neutral lipids, e.g. for identifying differentiation inducing substances. Thus, an effective, time-equivalent, metabolic monitoring can be integrated into the trials.

In comparison to step e) the method can be performed as end-point measurement or can be implemented in defined kinetics by set medium changes and measurements prior addition/after removal of media. To reduce effects due to cell proliferation, cell density as well as cell culture techniques should be chosen individually for every cell line (see application example).

With another embodiment the underlying task of the present invention is solved by the utilization of the markers lactate and neutral lipids to identify compounds inducing (re-)differentiation in a tumour cell. With regards to the detection of the markers we refer to the invented process.

The method of the present invention is in a preferred embodiment therefore characterised by the combined utilisation of the markers lactate and neutrals lipids for the identification of compounds inducing (re-)differentiation in un- or dedifferentiated cells, especially tumour cells. Especially preferred it is characterised by quantification of lactate concentration (in bicarbonate buffered cell culture medium incubated in an appropriate constant CO₂-atmosphere) by measuring the pH-dependent change of optical phenol red absorption and by quantification of neutral lipids using an appropriate neutral lipid staining (fluorescent) dye (e.g. BODIPY® 493/503).

The following application example further describes the tasks underlying the present invention in a non-limiting manner:

### EXAMPLE

To provide a homogenous, confluent cell layer, cells (cell lines: A549, MDA-MB-231 and PANC-1) were seeded with defined density and cultured for 24 h in a 384-well microtiter plate. The density of the cells is chosen in a way to reduce cell proliferation via growth arrest. In the following step, the medium is changed by centrifugation and the compound of interest is added. In case of MDA-MB-231 cells Na-Butyrate was added, in case of A549 cells Na-Butyrat and dexamethasone (DM) was added. The differentiation inducing effect of Na-Butyrate (including DM in case of A549) is known.

It might be necessary for some cell lines to change to serum-free media after the initial incubation of 24 h for a cell line dependent amount of time. Only afterwards, the addition of the compound of interest takes place. The compound's incubation time depends on the chosen tumour model and should be individually adjusted. If necessary, e.g. at incubation times from 24 h to 168 h, one/several medium changes with optional repeated additions of the compound of interest can be performed depending on the respective cell line.

Upon the expiry of the compound of interest's incubation time (from approx. 48 h to 168 h depending on the cell line) lactate concentration is determined in the supernatant. Therefore, changes in the pH-value of the samples are measured, using phenol red as indicator, and correlated to lactate concentration. For this purpose, the microtiter plate is analysed through the confluent cell layer.

Subsequently, a medium change via centrifugation was performed in order to remove the media completely. Any remaining media might have negatively affected the following measurement of the second marker neutral lipids.

After the complete exchange of the media, the fluorescent ligand BODIPY® 493/503 was added and incubated for 1 h following the removal of the dye containing media via centrifugation. The concentration of neutral lipids was determined via fluorescence measurement.

Substances inducing (re-)differentiation lead to a decrease of the first marker lactate and an increase in the second marker neutral lipids. In order to analyse both markers in a positive way, the first marker is mathematically converted into positive values, meaning high values of marker 1 now represent a high reduction of marker 1. In comparison to the mean values of untreated cells various compounds can be examined, analysed and evaluated with regard to their (re-)differentiation inducing abilities.

Directly following the measurement of both markers the cell viability of adherent cells is determined via absorption measurement in a microplate-reader. Whereby, the execution of the measurement is independent from the previously conducted measurements and is not further affected by previously added fluorescent ligands.

1. Method for the identification of compounds inducing (re-)differentiation in non- or dedifferentiated cells, comprising:
a) provision of a cell culture sample consisting of dedifferentiated and/or undifferentiated tumour cells,
b) bringing the compound of interest into contact with the cell culture sample,
c) following the determination of the relative concentration of a first marker lactate in contrast to untreated cells, and
d) following the determination of the relative concentration of a second marker neutral lipids in contrast to untreated cells,
wherein steps c) and d) may be performed in reverse order.

2. Method according to claim 1, characterised by lipid-free or lipid-reduced cultivation of the cell culture sample

3. Method according to claim 1, characterised by cultivating the cell culture sample under aerobic conditions

4. Method according to claim 1, which further includes:
c) determination of the amount of viable adherent cells via quantification of absorbance after centrifugation, and/or
f) determination of complete induction of apoptosis in the cells among a complete signal reduction to background level

5. Method according to claim 4, for utilisation as viability test and/or for utilisation as test for apoptosis.

6. Method according to claim 1, characterised by cultivating the cell culture sample in the presence of insulin.

7. Method according to claim 1, characterised by cultivating (tumour) cell culture sample either negative for/or low expressing insulin receptor isotype B or possessing a higher expression ratio of insulin receptor isotype A/B than differentiated cells in the presence vs. absence of insulin.

8. Method according to claim 1, characterised by a change in one or both of the first and second marker(s) in a presence vs. absence of insulin towards the direction of an anabolic change in the cell's metabolism enabling the analysis of functional insulin receptor subtype B expression and ratio of subtype A to B towards a relative higher subtype B expression, respectively.

9. Method according to claim 1 further, characterised by cultivating a breast cell culture sample negative for or poorly expressing prolactin receptor in a presence vs. absence of prolactin.

10. Method according to claim 1, characterised by a change in one or both of the first and second marker(s) in a presence vs. absence of prolactin towards the direction of an anabolic change in the cell's metabolism enabling the analysis of functional prolactin receptor expression.

11. Method according to claim 1, characterised by the provision of the cell culture sample in a microtiter plate.

12. Method according to claim 1, characterised by a gas permeable foil sealing the microtiter plate.

13. Method according to claim 1, characterised by quantification of lactate concentration in bicarbonate buffered cell culture medium incubated in an appropriate CO₂-atmosphere by measuring the pH-dependent change of optical phenol red absorption.

14. Method according to claim 1, characterised by single/multiple media and/or washing buffer removal from the cell culture sample via centrifugation prior to addition of new media for further culturing or measurement of marker in step 1d).

15. Method according to claim 1, characterised by performing single/multiple changes in media and/or washing buffer via partial device (A) and (B).

16. Method according to claim 1, characterised by the combined utilisation of the markers lactate and neutrals lipids for the identification of compounds inducing (re-)differentiation in un- or dedifferentiated cells, especially tumour cells.

17. Method according to claim 1, characterised by quantification of lactate concentration in bicarbonate buffered cell culture medium incubated in an appropriate constant CO₂-atmosphere by measuring the pH-dependent change of optical phenol red absorption and by quantification of neutral lipids using a neutral lipid staining dye.

18. Use of the markers lactate and neutrals lipids for the identification of compounds inducing (re-)differentiation in un- or dedifferentiated cells, especially tumour cells, wherein the markers are used combined.

19. A vessel for the removal of liquids from cells via centrifugation (partial device (A)) characterized by comprising:
4 side walls (a₁, a₂, b₁, b₂), where the two opposing side walls have the same length (lₐ, l_{b}), so that a rectangular shape is obtained,
a flat bottom (c) being connected in such a way with each of the side walls over all of the connecting area in a liquid proof way,
each of the side walls having a protrusion (dₐ, d_{b}) directed towards the inside of the vessel,
2 of the 4 side walls being opposite to each other having recesses (c₁, c₂) positioned in the middle of the lengths lₐ of the side wall on the upper surface of the respective side wall.

20. A microtiter plate (partial device (B)) for culturing cells enabling addition of liquid via centrifugation, said plate comprising a surface and wells, said wells being tapered towards the bottom where an opening, is present.

21. Microtiter plate according to claim 20 wherein the diameter of the opening is proportional to the surface tension of a liquid inside each of the wells and adjusted in such way that liquid can escape only under influence of forces stronger than the gravitational force.
